(19) United States
(12) Patent Application Publication
(10) Pub. No.: US 2020/0030137 A1
(43) Pub. Date: Jan. 30, 2020

(54) ORTHOPEDIC KNEE PAD

(71) Applicant: Dana Lavallet, Birmingham, AL (US)

(72) Inventor: Dana Lavallet, Birmingham, AL (US)

(21) Appl. No.: 16/043,388

(22) Filed: Jul. 24, 2018

(51) Int. Cl. A61F 7/02; A61F 7/08; A61F 5/01

(52) U.S. Cl. CPC A61F 7/02; A61F 7/08; A61F 2007/023; A61F 2007/0231; A61F 2005/0172; A61F 5/0123

(57) ABSTRACT

An orthopedic knee pad for secure placement between a user's knees that includes: a first side; a second side a plurality of thermal pads on the first side; and a means to secure the orthopedic knee pad in place during use. The means to secure may include hook and loop connectors on the first side. Preferably, the thermal pads include pockets adapted to receive thermal packs, where the thermal packs provide a cooling or warming sensation for the user. The means to secure may further include a plurality of tie strings at a first end of the orthopedic knee pad.

### BACKGROUND OF THE INVENTION

### Field of Invention

The present invention relates to an orthopedic pad used for placement in between the knees of a user during sleep.

### Description of Related Art

Many times individuals are injured, where they may sprain or otherwise injure a bone or joint. These injuries can cause throbbing pain and inflammation. The pain can be particular irritating at night when some is trying to sleep. One approach to provide comfort is the placement of a pillow between the knees. However, the pillow may not remain stable over the night. Although some pillows of the prior art have been designed to address this application, the pillows fail to address this stability issue. Thus, if the pillow becomes ajar then the user's sleep may be abruptly interrupted. Further in certain instances it is advisable to apply heat or cold to encourage healing and to reduce the stiffness of such joints.

The application of cold or heat therapy in conjunction with a pillow or pad can be difficult in certain instances depending on the location of the injury. Further application of such therapy is sometimes necessary prior to an individual going to sleep or as they fall asleep to relieve pain to encourage a relaxed state. As such, a pad or devices applied to an injured area may easily slide off during a person's sleep cycle. As a result, it would be advantageous to have a device that may be secured to an individual's knee that can remain secure while lounging or sleeping.

### SUMMARY OF THE INVENTION

The present invention relates to an orthopedic pad for application between a user's knees that includes: a first side; a second side a plurality of thermal pads on the first side; and a means to secure the orthopedic pad in place during use. The means to secure may include hook and loop connectors on the first side. Preferably, the thermal pads include pockets adapted to receive thermal packs, where the thermal packs provide a cooling or warming sensation for the user. The means to secure may further include a plurality of tie strings at a first end of the orthopedic pad.

### BRIEF DESCRIPTION OF DRAWINGS

FIG. 1 depicts an inside view of an orthopedic pad according to the present invention.

FIG. 2 depicts an outside view of an orthopedic pad according to the present invention.

### DETAILED DESCRIPTION

The present invention relates to an orthopedic knee pad to secure between an individual's knees while sleeping. The orthopedic knee pad according to present invention includes a first side and a second side, wherein the first side includes a plurality of thermal pads that may be optionally used by a user. A second side is provided, which is an outside surface of the orthopedic knee pad. The thermal pads of the first side are adapted to receive thermal packs, which generate either cold or warm therapy for application as desired by the user. Most importantly, the pad according to the present invention also uses means to attach and secure the pad into a stationary position so that pad remains stable between the knees while sleeping.

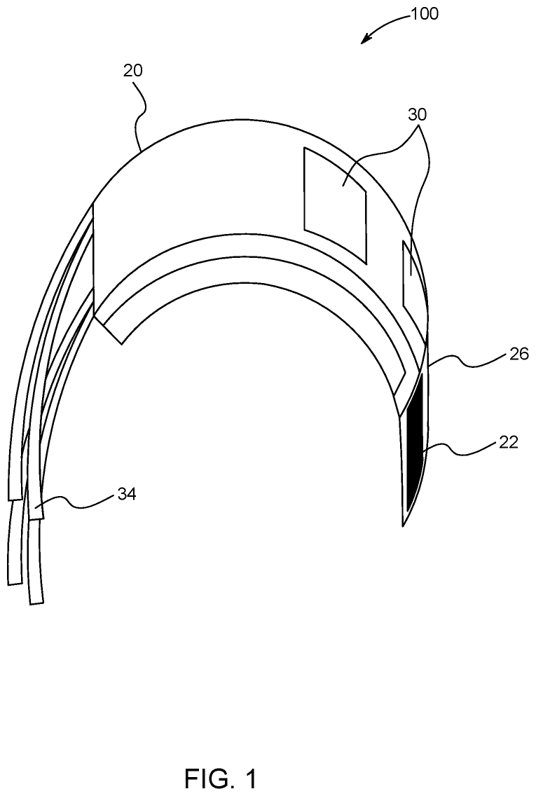

In reference to FIG. 1 an orthopedic knee pad 100 according to the present invention is depicted. This orthopedic knee pad 100 is shown on a first side 20, an inside surface, in FIG. 1. The first side 20 includes two thermal pads 30 that include pockets that may receive thermal packs. A thermal pack may be cool or hot to transfer either a cooling or heating sensation as desired by the user. These packets may be optionally secured in the thermal pads 30 and the orthopedic knee pad is secured across a joint or a limb of an individual as desired. Hook and loop fasteners 22 are provided as a first securing means to secure the orthopedic pad 100 in a stationary position. FIG. 1 shows a connecting end 26 that includes hook and loop connectors to join with the hook and loop connectors 22 on the first side of the orthopedic pad 100. The opposing end of the orthopedic pad 100 includes string ties 34 that are further used to secure the orthopedic knee pad 100 in a stationary position between the knees of a user.

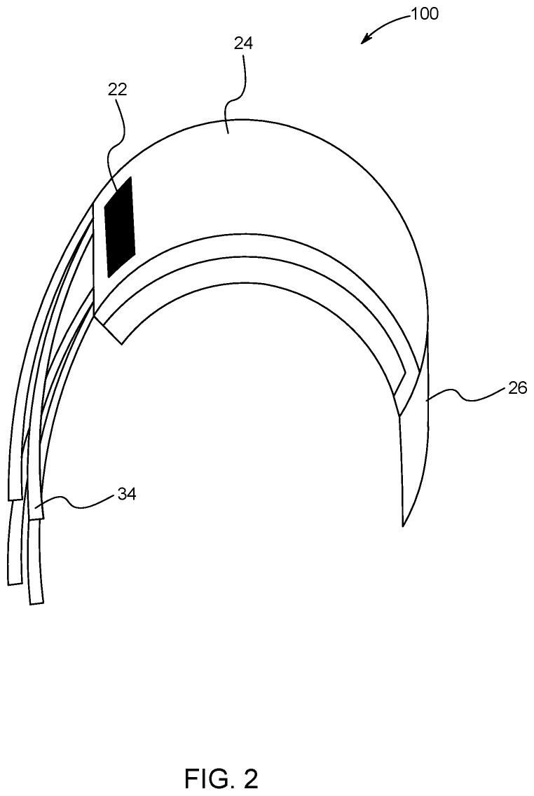

FIG. 2 depicts a second side 24 of the orthopedic knee pad 100. The second side 24 is the outside surface of the orthopedic knee pad 100 and includes a generally smooth surface to allow for easy application of the orthopedic knee pad 100 onto the surface of a first knee. Once the knee pad 100 is secured to a first knee, a user may use the pad throughout the night without concern of the knee pad 100 becoming unstable during use. The foregoing descriptions of specific embodiments of the present invention have been presented for purposes of illustration and description. They are not intended to be exhaustive or to limit the invention to the precise forms disclosed, and obviously many modifications and variations are possible in light of the above teaching. The exemplary embodiment was chosen and described in order to best explain the principles of the invention and its practical application, to thereby enable others skilled in the art to best utilize the invention and various embodiments with various modifications as are suited to the particular use contemplated.

What is claimed is:

1. An orthopedic knee pad for secure placement between a user's knees comprising:
a. a first side;
b. a second side
c. a plurality of thermal pads on the first side; and
d. a means to secure the orthopedic pad in place during use.

2. The orthopedic knee pad according to claim 1, where the means to secure includes hook and loop connectors on the first side.

3. The orthopedic knee pad according to claim 1, where the thermal pads include pockets adapted to receive thermal packs.

4. The orthopedic knee pad according to claim 3, where the thermal packs provide a cooling sensation for the user.

5. The orthopedic knee pad according to claim 3, where the thermal pack provide a warming sensation for the user.

6. The orthopedic knee pad according to claim 2, where the means to secure further includes a plurality of tie strings at a first end of the orthopedic pad.
